(19) United States
(12) Patent Application Publication
(10) Pub. No.: US 2026/0283461 A1
(43) Pub. Date: Sep. 24, 2026

(54) OCULAR TOMOGRAPHIC IMAGE GENERATION METHOD

(71) Applicant: DEEPEYEVISION INC., Shimotsuke-shi, Tochigi (JP)

(72) Inventor: Hidenori TAKAHASHI, Shimotsuke-shi, Tochigi (JP)

(21) Appl. No.: 19/474,308

(22) PCT Filed: Jun. 25, 2024
(86) PCT No.: PCT/JP2024/022969

§ 371 Date: Oct. 10, 2025

(30) Foreign Application Priority Data
Jul. 26, 2023 (JP) 2023-121603

(51) Int. Cl. A61B 3/00; A61B 3/10; G06T 7/00; G06T 7/521

(52) U.S. Cl. CPC A61B 3/0025; A61B 3/1005; A61B 3/102; G06T 7/0012; G06T 7/521; G06T 2207/10101; G06T 2207/20081; G06T 2207/30041

(57) ABSTRACT

To provide an ocular tomographic image generation method that enables an ocular tomographic image to be acquired with a simple configuration of an optical system even when a distance between a test eye and an ophthalmic analysis device is long. The ocular tomographic image generation method including: a first step of acquiring depth information of an intraocular structure of the test eye using a range sensor; a second step of acquiring a plurality of pieces of depth information while changing relative positions of the test eye and the range sensor; a third step of specifying a three-dimensional position in the eyeball of the depth information; and a fourth step of generating an ocular tomographic image by compositing the plurality of pieces of depth information based on the position information.

### CROSS-REFERENCES TO RELATED APPLICATIONS

The present application is based on Japanese Patent Application No. 2023-121603 filed on Jul. 26, 2023, the contents of which are incorporated herein by reference.

### TECHNICAL FIELD

The present disclosure relates to an ocular tomographic image generation method.

### BACKGROUND ART

It is very important to generate an ocular tomographic image in terms of diagnosis of fundus disease and risk assessment of glaucoma attack. Examples of a typical method for generating an ocular tomographic image include usage of optical coherence tomography (OCT). The OCT can acquire an ocular tomographic image by emitting near-infrared light to a test eye and analyzing reflection from an intraocular structure.

For an ocular disease such as a fundus macular disease or glaucoma, long-term follow-up observation is often required. Thus, various ocular tomographic image generation methods are developed for the purpose of efficiently and preferably performing follow-up observation on the test eye. For example, Patent Literature 1 discloses a predetermined ophthalmic analysis device for obtaining analysis results of tomographic images of the test eye acquired on different days by an ophthalmic optical coherence tomography device and outputting statistical information formed from time-series data of the analysis results.

### CITATION LIST

### Patent Literature

Patent Literature 1: Japanese Patent Laid-Open No. 2014-83266

### SUMMARY OF INVENTION

In a case of generating an ocular tomographic image using such an ophthalmic analysis device, it is necessary to perform intraocular observation in a state where a distance between a test eye and a device is very close such that a chin of a patient is placed on a chin rest of a stand-mounted device. Additionally, in a case of performing intraocular observation, an ophthalmic analysis device installed in a hospital is required for medical examination, so that telemedicine and the like cannot be supported.

Thus, the present invention has been made in view of the above problems and has an object to provide an ocular tomographic image generation method that enables an ocular tomographic image to be acquired with a simple configuration of an optical system even when the distance between the test eye and the ophthalmic analysis device is long.

To solve the above problems, the present inventors have conducted active studies. As a result, the present inventors have found that the problems can be solved by the following ocular tomographic image generation method.

In other words, the ocular tomographic image generation method according to an aspect of the present disclosure includes a first step of acquiring depth information of an intraocular structure of the test eye using a range sensor, a second step of acquiring a plurality of pieces of the depth information while changing relative positions of the test eye and the range sensor, a third step of specifying a three-dimensional position in the eyeball of the depth information, and a fourth step of generating an ocular tomographic image by compositing the plurality of pieces of depth information based on the position information.

At the third step, the three-dimensional position of the depth information may be specified based on an incidence angle of incident light from the range sensor with respect to a corneal vertex of the test eye and/or a thickness of a retina.

At the fourth step, the ocular tomographic image may be generated by a mathematical model trained by machine learning or deep learning.

At the fourth step, the ocular tomographic image may be complemented by using a standard tomographic image.

At the fourth step, the plurality of pieces of depth information acquired at the second step may be used as training data for the mathematical model.

At the first step, resolution processing may be performed using adaptive optics.

The ocular tomographic image generation method may further include a disease risk information acquisition step of acquiring information about presence/absence of disease risk by inputting the depth information to a mathematical model trained by machine learning or deep learning using ocular tomographic image data related to an ocular disease as training data.

The ocular tomographic image generation method may further include a display step of displaying the depth information.

The range sensor may be a distance measuring unit of a mobile terminal.

The range sensor may be a distance measuring unit of a stationary device.

The range sensor may be a distance measuring unit of a head-mounted device.

The present disclosure can provide an ocular tomographic image generation method that enables an ocular tomographic image to be acquired with a simple configuration of an optical system even when a distance between a test eye and an ophthalmic analysis device is long.

### BRIEF DESCRIPTION OF DRAWINGS

FIG. 1 is an example of a diagram illustrating an aspect of acquiring depth information of an intraocular structure of a test eye using a range sensor.

FIG. 2 is an example of a diagram illustrating an example of depth information related to a cornea, an iris, and a retina.

FIG. 3 is an example of a diagram illustrating an aspect of acquiring a plurality of pieces of depth information of the intraocular structure while changing relative positions of the test eye and the range sensor.

FIG. 4 is an example of a diagram illustrating change in scenery reflected in the test eye, change in a corneal limbus of the test eye, movement of a pupil center in the corneal limbus, or change in a pupil border according an embodiment of the present disclosure.

FIG. 5 is an example of a diagram illustrating part of an ocular tomographic image including a cornea and an iris among ocular tomographic images obtained by an ocular tomographic image generation method according to the present embodiment.

FIG. 6 is an example of a diagram illustrating part of an ocular tomographic image including a retina among the ocular tomographic images obtained by the ocular tomographic image generation method according to the present embodiment.

FIG. 7 is a schematic diagram illustrating an example of using a mobile terminal according to an embodiment of the present disclosure as an ocular tomographic image generation device.

FIG. 8 is a schematic diagram illustrating an example of using a stationary device according to an embodiment of the present disclosure as the ocular tomographic image generation device.

FIG. 9 is a schematic diagram illustrating an example of using a head-mounted device according to an embodiment of the present disclosure as the ocular tomographic image generation device.

FIG. 10 is a flowchart related to implementation of the ocular tomographic image generation device according to an embodiment of the present disclosure.

FIG. 11 is a hardware configuration of the ocular tomographic image generation device according to an embodiment of the present disclosure.

FIG. 12 is a functional block configuration of the ocular tomographic image generation device according to an embodiment of the present disclosure.

### DESCRIPTION OF EMBODIMENTS

The following describes an embodiment of the present invention with reference to the attached drawings. The following embodiment is provided for facilitating understanding of the present invention, and is not to be construed as limiting the present invention. The present invention can be variously modified without departing from the gist of the invention. Furthermore, those skilled in the art can adopt embodiments obtained by replacing elements described below with equivalents and such embodiments are also included in the scope of the present invention.

### 1. Ocular Tomographic Image Generation Device

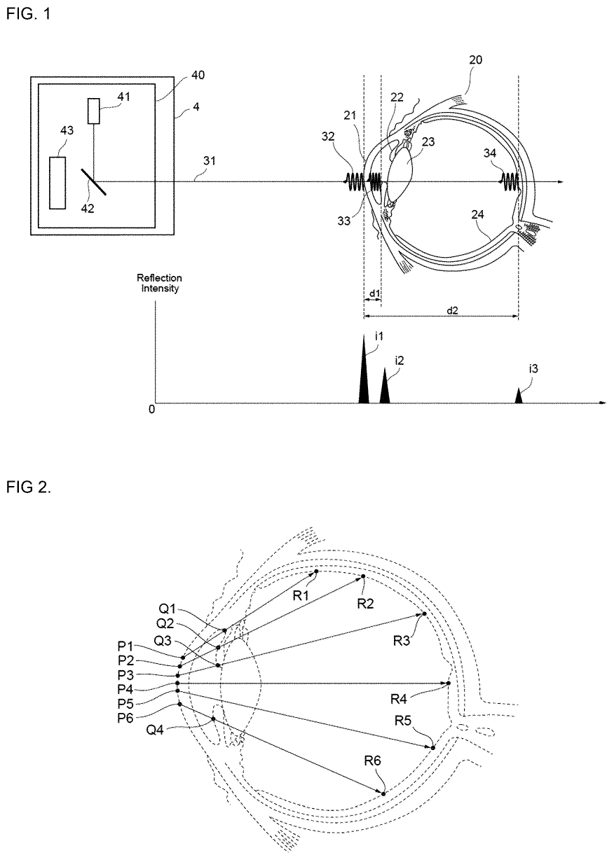

FIG. 1 is a diagram illustrating an example of a method of acquiring depth information of an intraocular structure of a test eye by an ocular tomographic image generation device 4. The ocular tomographic image generation device 4 according to the present embodiment is a device that acquires depth information of an intraocular structure of a test eye 20 by causing incident light 31 to be incident on the test eye 20 using a light source 41 and a beam splitter 42 of a range sensor 40, and detecting, by a detector 43, various kinds of reflected light derived from the intraocular structure such as reflected light 32 from a cornea 21, reflected light 33 from an iris 22, and reflected light 34 reflected by a retina 24.

### <Hardware Configuration>

FIG. 11 is a diagram illustrating an example of a hardware configuration of the ocular tomographic image generation device 4. The ocular tomographic image generation device 4 includes a processor 11 such as a Central Processing Unit (CPU) and a Graphical Processing Unit (GPU), a storage device 12 such as a memory, Hard Disk Drive (HDD), and/or a Solid State Drive (SSD), a communication Interface (IF) 13 that performs wired or wireless communication, an input device 14 that receives an input operation, and an output device 15 that outputs information. The input device 14 is, for example, a keyboard, a touch panel, a mouse, and/or a microphone. The output device 15 is, for example, a display, a touch panel, and/or a speaker.

In the present embodiment, the range sensor 40 may be configured to be included in the input device 14. Alternatively, the range sensor 40 may be configured to be included in an external apparatus of the ocular tomographic image generation device 4 so that depth information acquired by the range sensor 40 via a communication line and the like is input through the input device 14.

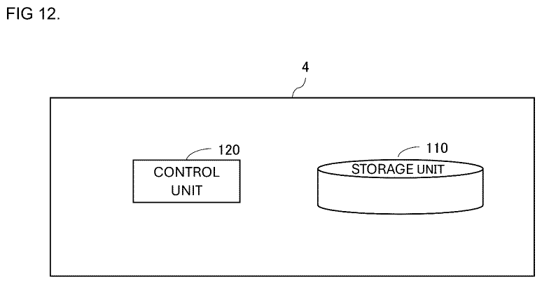

FIG. 12 is a diagram illustrating an example of a functional block configuration of the ocular tomographic image generation device 4. The ocular tomographic image generation device 4 includes a storage unit 110 and a control unit 120. The storage unit 110 can be implemented by using the storage device 12 included in the ocular tomographic image generation device 4. The control unit 120 can be implemented by executing a program stored in the storage device 12 by the processor 11 of the ocular tomographic image generation device 4. The program can be stored in a storage medium. The storage medium in which the program is stored may be a non-transitory computer-readable medium. The non-transitory storage medium is not particularly limited, but may be a storage medium such as a USB memory or a CD-ROM, for example.

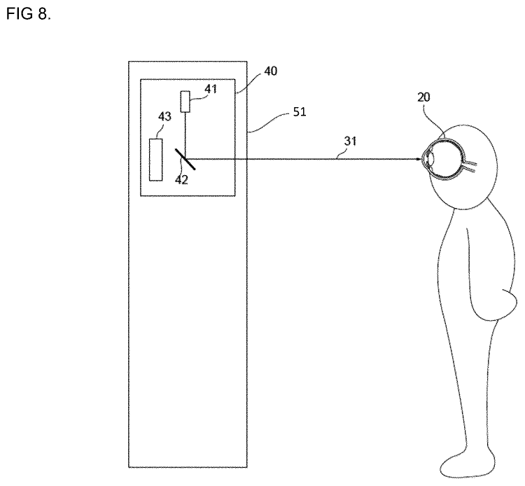

FIG. 7 is a schematic diagram illustrating an example of using a mobile terminal 50 as the ocular tomographic image device 4. By using the mobile terminal 50 as the ocular tomographic image device 4, an ocular tomographic image can be generated with a simpler configuration of an optical system. For convenience of description, FIG. 7 illustrates the mobile terminal 50 in an enlarged manner relative to an actual size. The range sensor 40 is not particularly limited, but a sensor of the mobile terminal 50 may be used, for example. The mobile terminal 50 is not particularly limited, but examples thereof include information processing devices such as a smartphone, a tablet computer, a notebook computer, or a workstation computer, for example. Herein, the terminal means a piece of equipment capable of performing a key role in conducting communications with other equipment by being connected to a circuit or a network.

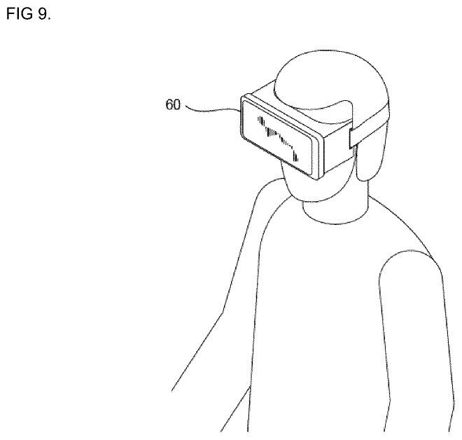

FIG. 8 is a schematic diagram illustrating an example of using a stationary device 51 as the ocular tomographic image generation device 4. By using the stationary device 51 as the ocular tomographic image device 4, an ocular tomographic image can be generated with a simpler configuration of an optical system. The range sensor 40 is not particularly limited, but a range sensor of the stationary device 51 is used, for example. In a case of using the stationary device 51 as the ocular tomographic image generation device, when a subject moves while facing the stationary device 51, relative positions of the test eye 20 and the range sensor 40 can be easily changed. Additionally, second step S102 (described later) can be performed for a certain time without any operation performed by the subject.

The stationary device 51 is not particularly limited, but may be a smart mirror, for example. By using the smart mirror, the subject can be naturally photographed in daily life without consciousness. Herein, the smart mirror means a mirror-type terminal capable of being connected to the Internet, and including a mirror that can reflect a figure of the subject and a range sensor.

FIG. 9 is a schematic diagram illustrating an example of using a head-mounted device 60 as the ocular tomographic image generation device 4. By using the head-mounted device 60 as the ocular tomographic image generation device 4, an ocular tomographic image can be generated with a simpler configuration of an optical system. The range sensor 40 is not particularly limited, but a range sensor incorporated in the head-mounted device 60 is used, for example. In a case of using the head-mounted device 60 as the ocular tomographic image generation device 4, as illustrated in FIG. 9, first step S101 and second step S102 (described later) can be easily performed irrespective of an action of the subject.

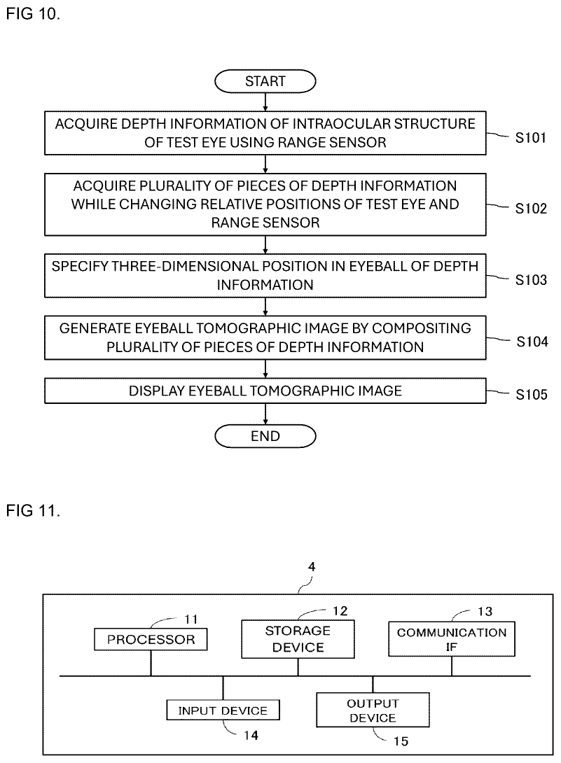

FIG. 10 is a flowchart illustrating steps related to implementation of the ocular tomographic image generation device 4 according to an embodiment of the present invention, and each of the steps is performed by the control unit 120 of the ocular tomographic image generation device 4. The following describes each of the steps in detail with reference to FIG. 10.

### 2. Ocular Tomographic Image Generation Method

In FIG. 10, the ocular tomographic image generation device 4 includes first step S101 of acquiring the depth information of the intraocular structure of the test eye, second step S102 of acquiring a plurality of pieces of the depth information while changing relative positions of the test eye and the range sensor, third step S103 of specifying a three-dimensional position in an eyeball of the depth information, and fourth step S104 of generating an ocular tomographic image by compositing the plurality of pieces of depth information, and generates an ocular tomographic image of the test eye 20 by performing these steps. The ocular tomographic image generation device 4 may further include display step S105. Furthermore, other steps may be included as needed.

### 2.1. First Step S101

As illustrated in FIG. 1, first step S101 is a step of acquiring the depth information of the intraocular structure of the test eye 20 using the range sensor 40. At first step S101, the incident light 31 is caused to be incident on the test eye 20 using the light source 41 and the beam splitter 42 of the depth sensor 40 in the ocular tomographic image generation device 4, and reflected light from the intraocular structure is detected by the detector 43. FIG. 1 illustrates the fact that the reflected light 32 from the cornea 21, the reflected light 33 from the iris 22, and the reflected light 34 reflected by the retina 24 can be detected.

Herein, in FIG. 1, d1 indicates an anterior chamber depth, and d2 indicates an ocular axial length. d1 is information useful for diagnosis of glaucoma and the like, and d2 is information useful for diagnosis of myopia and the like.

Herein, the range sensor means a sensor that measures a distance. The range sensor is not particularly limited, but may be a range sensor using optics (LiDAR), a range sensor using radio waves, and a range sensor using ultrasonic waves. Among these, it is preferable to use the LiDAR in terms of easily acquiring the depth information. The LiDAR is not particularly limited, but may be a ToF type LiDAR or a FMCW type LiDAR, for example.

Among these, it is preferable to use the FMCW type LiDAR in the present embodiment. The FMCW type LiDAR emits a laser beam that is continuously shifted between different frequencies, so that when a detector collects reflected light and measures a reflection time, a specific frequency pattern and other light sources can be distinguished from each other, and a high-speed correct operation is enabled under all irradiation conditions.

Due to this, even in a case where light from a surrounding light source and reflected light thereof can easily enter the detector because of a long distance between the test eye and the range sensor, the depth information tends to be acquired more accurately. Also, in a case of reflection intensity of very weak reflected light derived from the intraocular structure as in the present embodiment, the depth information tends to be acquired more accurately.

The intraocular structure means overall structure inside the eyeball, and is not particularly limited. For example, a ciliary body, a zonule of Zinn, a choroid, and a sclera are exemplified in addition to the cornea 21, the iris 22, and the retina 24 illustrated in FIG. 1.

The depth information means information about a distance from the range sensor located at a predetermined relative position with respect to the test eye to the intraocular structure. The depth information can be obtained from reflected light, which is obtained when incident light emitted from the range sensor located at the predetermined relative position with respect to the test eye is reflected and discharged by the intraocular structure.

In FIG. 2, P1, P2, P3, P4, P5, and P6 indicate examples of the depth information related to the cornea, Q1, Q2, Q3, and Q4 indicate examples of the depth information related to the iris, and R1, R2, R3, R4, R5, and R6 indicate examples of the depth information related to the retina.

Typically, in a case of detecting reflected light inside a substance, intensity of reflected light reflected from a structure in the vicinity of a surface of the substance tends to be maximum, and the intensity of reflected light from the internal structure tends to become smaller as advancing toward the inner part of the substance. In FIG. 1, i1 indicates intensity of the reflected light 32 of the cornea 21, i2 indicates intensity of the reflected light 33 from the iris 22, and i3 indicates intensity of the reflected light 34 from the retina 24. As the incident light moves from the surface of the test eye to the inner part, the intensity of reflected light from the intraocular structure tends to be smaller.

At first step S101, an ocular tomographic image can be generated even when the range sensor 40 of the ocular tomographic image generation device 4 is distant from the test eye 20. The distance between the range sensor 40 and the test eye 20 is not particularly limited, but is preferably equal to or larger than 3 cm, more preferably equal to or larger than 5 cm, even more preferably equal to or larger than 10 cm, yet more preferably equal to or larger than 15 cm, further more preferably equal to or larger than 20 cm, and particularly preferably equal to or larger than 30 cm in terms of easily generating an ocular tomographic image.

A lower limit of the distance between the range sensor 40 and the test eye 20 is not particularly limited, but may be 1 mm, 3 mm, 5 mm, or 1 cm, for example.

An upper limit of the distance between the range sensor 40 and the test eye 20 is not particularly limited, but may be 10 m, 5 m, 3 m, 1 m, or 50 cm, for example.

At first step S101, resolution processing may be performed by using adaptive optics. Specifically, for example, a range sensor to which adaptive optics is applied may be used.

Herein, the adaptive optics means a technique of obtaining a high-resolution image by removing wave front aberration.

### 2.2. Second Step S102

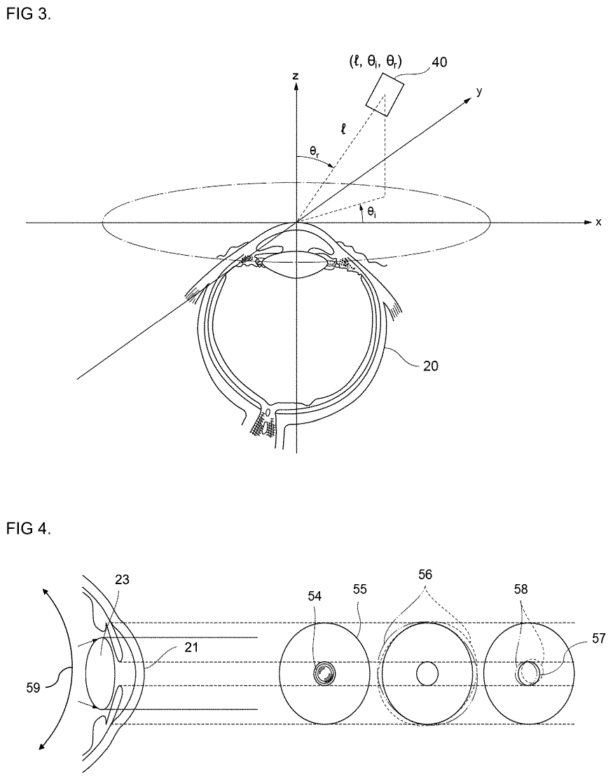

As illustrated in FIG. 3, second step S102 is a step of acquiring a plurality of pieces of depth information while changing relative positions (1, θᵢ, θᵣ) of the test eye 20 and the range sensor 40.

Details will be described below.

As means for changing the relative positions (1, θᵢ, θᵣ) of the test eye 20 and the range sensor 40, a conventional well-known method can be used. Such a method is not particularly limited. For example, orientation of the test eye 20 may be changed without changing the position of the range sensor 40, or the position of the range sensor 40 may be changed without changing the orientation of the test eye 20.

As means for changing the orientation of the test eye 20 is not particularly limited. For example, fixation target projection means for presenting a plurality of fixation targets may be used. The fixation target projection means includes a fixation lamp that emits visible light, and can presents the plurality of fixation targets to the subject by lighting the fixation lamp, and guide the test eye 20 to view an optional direction.

The number of pieces of the depth information to be acquired is used for image generation step S104 (described later), so that it is not particularly limited. For example, the number of pieces of the depth information is preferably equal to or larger than 30, more preferably equal to or larger than 100, and even more preferably equal to or larger than 300. Herein, the number of pieces of the depth information is counted as one for each of the relative positions (1, θᵢ, θᵣ) of the test eye 20 and the range sensor 40.

2.3. Third step S103

Third step S103 is a step of specifying a three-dimensional position in the eyeball of the depth information.

Third step S103 may be performed based on the pieces of depth information obtained by changing the relative positions (1, θᵢ, θᵣ) of the test eye 20 and the range sensor 40. As such a method for specifying the three-dimensional position, a conventional well-known method can be used. The method is not particularly limited, but Structure from Motion (SfM) can be exemplified, for example.

At third step S103, the three-dimensional position of the depth information may be specified based on an incidence angle of the incident light 31 from the range sensor 40 with respect to the corneal vertex of the test eye 20 and/or a thickness of the retina 24.

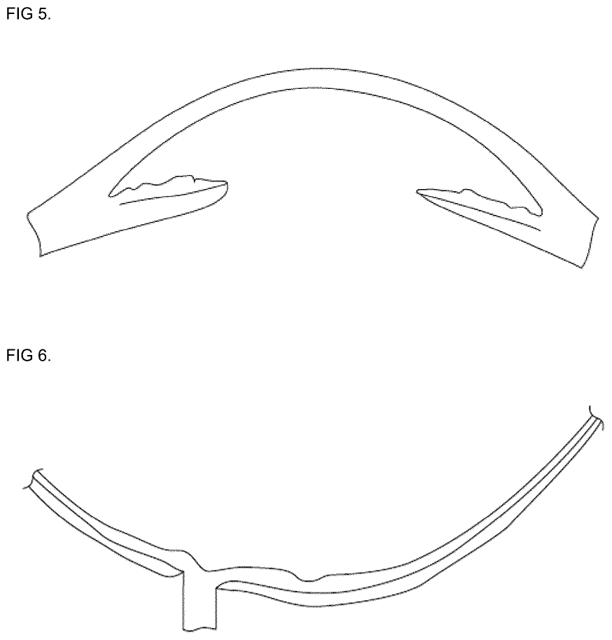

At third step S103, as illustrated in FIG. 4, it is possible to use pieces of information in combination, the information including change in scenery 54 reflected in the test eye 20 such as movement of scenery reflected in a pupil of the test eye 20, change in a corneal limbus 55 of the test eye 20, movement of a pupil center in the corneal limbus 55, or change in a pupil border. Specifically, accuracy in specification of the three-dimensional position of the depth information can be improved by a method used for specifying the three-dimensional position of the depth information by detecting change in the scenery 54 reflected in the test eye 20 caused by eyeball rotational movement 59 of the test eye 20, a method used for specifying the three-dimensional position of the depth information by detecting change 56 in the corneal limbus caused by the rotational movement 59 of the eyeball such as deformation of the corneal limbus 55 from a circular shape to a substantially elliptic shape corresponding to the rotational movement 59 of the eyeball, a method used for specifying the three-dimensional position of the depth information by detecting movement 58 of the pupil center in the corneal limbus caused by the rotational movement 59 of the eyeball, or detecting change in a pupil border 57 caused by the rotational movement 59 of the eyeball.

### 2.4. Fourth Step S104

Fourth step S104 is a step of compositing the plurality of pieces of depth information to generate an ocular tomographic image based on the pieces of depth information obtained at second step S102 and the three-dimensional position in the eyeball specified at three-dimensional position information specification step S103.

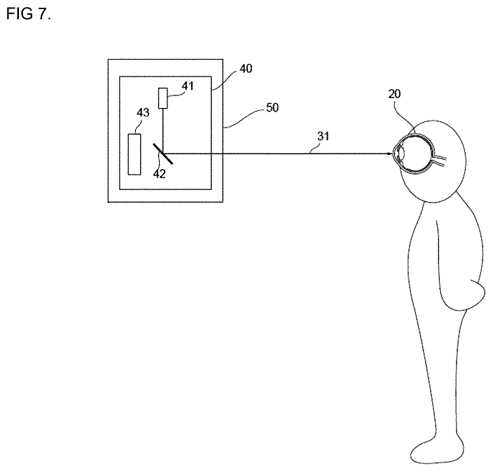

FIGS. 5 and 6 are examples of the ocular tomographic image obtained at the fourth step. FIG. 5 illustrates part of the ocular tomographic image including the cornea 21 and the iris 22, and FIG. 6 illustrates part of the ocular tomographic image including the retina.

At the fourth step, the ocular tomographic image may be generated by a mathematical model trained by machine learning or deep learning.

At the fourth step, the ocular tomographic image may be complemented by using a standard tomographic image. Herein, as a method for acquiring the standard tomographic image, a conventional well-known method can be used. The method for acquiring the standard tomographic image is not particularly limited, but an optical coherence tomography (OCT) can be used, for example.

Herein, training data for the mathematical model is not particularly limited, but it is possible to use the plurality of pieces of depth information acquired at the second step described above, for example. Due to this, the depth information corresponding to an unacquired region can be generated and complemented at second step S102 without using the standard tomographic image. Accordingly, the number of arithmetic operations by a computer is increased but the standard tomographic image for the test eye 20 is not required to be prepared, so that the ocular tomographic image can be easily generated.

Herein, a learning model trained by machine learning (hereinafter, referred to as a machine learning model) is an example of the mathematical model. The machine learning model includes a model including a predetermined model structure and parameters that vary due to learning processing, the model in which discrimination accuracy is improved when processing parameters thereof are optimized based on experience obtained from the training data. That is, the machine learning model is a model that learns optimum processing parameters by learning processing. As an algorithm of the machine learning model, for example, a support vector machine, logistic regression, random forest, a neural network, and the like can be used, and types thereof are not particularly limited. In terms of generating the ocular tomographic image even when an amount of the training data is small, the neural network is preferably used. The machine learning model that performs the learning includes a model that has performed some learning with the training data, and a model before learning.

### 2.5. Display Step S105

As illustrated in FIG. 10, display step S105 is a step of displaying the ocular tomographic image, and the ocular tomographic image generation device 4 in the present embodiment preferably includes display step S105. Due to this, intraocular information of the subject can be checked more easily.

### 2.6. Other Steps

The ocular tomographic image generation device 4 in the present embodiment may include other steps as needed in addition to the above-described steps. The other steps are not particularly limited, but a disease risk information acquisition step is exemplified, for example. The disease risk information acquisition step is a step of acquiring information about presence/absence of a disease risk by inputting the depth information to a mathematical model trained by machine learning or deep learning using the ocular tomographic image data related to an ocular disease as training data. This step can be used for easily finding an ocular disease such as glaucoma or myopia at an early stage.

### INDUSTRIAL APPLICABILITY

According to the present invention, without particular limitation, it is possible to easily acquire an ocular tomographic image used for ophthalmic examination using a mobile terminal and the like including a smartphone, for example. Thus, the method for acquiring the ocular tomographic image according to the present invention is expected to be widely spread in the world.

It also has great significance in clinical application. Specifically, in medical examination performed by a typical eye doctor, observation and diagnosis of a test eye are performed by using a stand-mounted device, but medical examination for an infant or a bedridden elderly person involves difficulties, and a skilled technique is required. However, in the present invention, the ocular tomographic image can be very easily generated by using a mobile terminal and the like irrespective of a posture of the subject, and it can be used for observation and diagnosis accordingly. Thus, the present invention is expected to be used for telemedicine in local regions or support for developing countries.

Furthermore, the present invention can be similarly used for observation and photographing of an eyeball of an animal. Particularly, it is possible to acquire an ocular tomographic image of a companion animal such as a pet and a large captive animal in a zoo whose test eye is particularly difficult to be brought closer to an ophthalmic diagnostic device, and ophthalmic findings of the above-described animal can be acquired.

Furthermore, it is expected that data is analyzed by AI as big data, and diagnostic accuracy of eye doctors is improved. Finally, it can be used as a self-diagnostic tool for a subject, and ophthalmic medical care itself can be further developed.

### REFERENCE SIGNS LIST

4 . . . ocular tomographic image generation device, 20 . . . test eye, 21 . . . cornea, 22 . . . iris, 23 . . . crystalline lens, 24 . . . retina, 31 . . . incident light, 32 . . . reflected light from cornea 21, 33 . . . reflected light from iris 22, 34 . . . reflected light from retina 24, 40 . . . range sensor, 41 . . . light source, 42 . . . beam splitter, 43 . . . detector, 50 . . . mobile terminal, 51 . . . stationary device, 60 . . . head-mounted device, i1 . . . reflection intensity of reflected light from cornea 21, i2 . . . reflection intensity of reflected light from iris 22, i3 . . . reflection intensity of reflected light from retina 24, P1, P2, P3, P4, P5, P6, Q1, Q2, Q3, Q4, R1, R2, R3, R4, R5, R6 . . . depth information, ▪. . . distance from corneal vertex to range sensor, θᵢ, θᵣ . . . azimuth angle of range sensor with respect to corneal vertex, 54 . . . scenery reflected in test eye, 55 . . . corneal limbus, 56 . . . change in corneal limbus, 57 . . . pupil border, 58 . . . movement of pupil center in corneal limbus, 59 . . . rotational movement of eyeball, 11 . . . processor, 12 . . . storage device, 13 . . . communication IF, 14 . . . input device, 15 . . . output device, 110 . . . storage unit, 120 . . . control unit, S101 . . . first step, S102 . . . second step, S103 . . . third step, S104 . . . fourth step, S105 . . . display step

1. An ocular tomographic image generation method comprising:
a first step of acquiring depth information of an intraocular structure of a test eye using a range sensor;
a second step of acquiring a plurality of pieces of the depth information while changing relative positions of the test eye and the range sensor;
a third step of specifying a three-dimensional position in an eyeball of the depth information; and
a fourth step of generating an ocular tomographic image by compositing the plurality of pieces of depth information based on the position information.

2. The ocular tomographic image generation method according to claim 1, wherein the three-dimensional position of the depth information is specified based on an incidence angle of incident light from the range sensor with respect to a corneal vertex of the test eye and/or a thickness of a retina at the third step.

3. The ocular tomographic image generation method according to claim 1, wherein the ocular tomographic image is generated by a mathematical model trained by machine learning or deep learning at the fourth step.

4. The ocular tomographic image generation method according to claim 1, wherein the ocular tomographic image is complemented by using a standard tomographic image at the fourth step.

5. The ocular tomographic image generation method according to claim 3, wherein the plurality of pieces of depth information acquired at the second step are used as training data for the mathematical model.

6. The ocular tomographic image generation method according to claim 1, wherein resolution processing is performed by using adaptive optics at the first step.

7. The ocular tomographic image generation method according to claim 1, further comprising a disease risk information acquisition step of acquiring information about presence/absence of a disease risk by inputting the depth information to a mathematical model that is trained by machine learning or deep learning using ocular tomographic image data related to an ocular disease as training data.

8. The ocular tomographic image generation method according to claim 1, further comprising a display step of displaying the depth information.

9. The ocular tomographic image generation method according to claim 1, wherein the range sensor is a distance measuring unit of a mobile terminal.

10. The ocular tomographic image generation method according to claim 1, wherein the range sensor is a distance measuring unit of a stationary device.

11. The ocular tomographic image generation method according to claim 1, wherein the range sensor is a distance measuring unit of a head-mounted device.

12. An ocular tomographic image generation system comprising a processor, wherein the processor is configured to:
acquire depth information of an intraocular structure of a test eye using a range sensor;
acquire a plurality of pieces of the depth information while changing relative positions of the test eye and the range sensor;
specify a three-dimensional position in an eyeball of the depth information; and
generate an ocular tomographic image by compositing the plurality of pieces of depth information based on the position information.

13. A program causing computer to execute:
acquiring depth information of an intraocular structure of a test eye using a range sensor;
acquiring a plurality of pieces of the depth information while changing relative positions of the test eye and the range sensor;
specifying a three-dimensional position in an eyeball of the depth information; and
generating an ocular tomographic image by compositing the plurality of pieces of depth information based on the position information.
